(19) United States
(12) Patent Application Publication
(10) Pub. No.: US 2008/0114027 A1
(43) Pub. Date: May 15, 2008

(54) Method for treating delirium

(76) Inventor: Kirk W. Johnson, Moraga, CA (US)

Correspondence Address: ROBINS & PASTERNAK LLP, 1731 EMBARCADERO ROAD, SUITE 230, PALO ALTO, CA, 94303, US

(21) Appl. No.: 11/982,714

(22) Filed: Nov. 2, 2007

Related U.S. Application Data
(60) Provisional application No. 60/857,887, filed on Nov. 9, 2006.

(51) Int. Cl. A61K 31/4745

(52) U.S. Cl. 514/303

(57) ABSTRACT

Methods for treating delirium, including postoperative delirium, sepsis-associated delirium, and drug or alcohol withdrawal-associated delirium with ibudilast are disclosed. Treatment may further comprise administration of one or more other agents, such as analgesics, anti-inflammatory and central nervous system (CNS)-partitioning phosphodiesterase inhibitors, antidepressants, neuroleptics, benzodiazepines, and procholinergic agents.

### CROSS-REFERENCE TO RELATED APPLICATIONS

This application claims the benefit under 35 U.S.C. §119(e)(1) to U.S. Provisional Application Ser. No. 60/857,887, which application is incorporated herein by reference in its entirety.

### FIELD OF THE INVENTION

The present invention relates generally to methods for treating delirium, including postoperative delirium, sepsis-associated delirium, and drug or alcohol withdrawal-associated delirium. In particular, the present invention pertains to methods for treating delirium, with ibudilast (also termed AV411 herein). Treatment may further comprise administration of one or more other agents, such as analgesics, anti-inflammatory and central nervous system (CNS)-partitioning phosphodiesterase inhibitors, antidepressants, neuroleptics, benzodiazepines, and procholinergic agents.

### BACKGROUND OF THE INVENTION

Delirium is a common medical complication occurring in about 15-20% of patients admitted to a hospital and with higher frequency in elderly patients or those with pre-existing cognitive impairment (Meagher BMJ 322:144-149, 2001). Patients particularly at risk for developing delirium include those experiencing surgical trauma, withdrawal from drugs or alcohol, metabolic disturbances, or sepsis. Symptoms of delirium include hallucinations, delusions, anxiety, memory impairment, and confusion.

Postoperative delirium commonly occurs following major surgeries and is associated with higher incidences of postoperative morbidity and mortality (Hala, Med Hypotheses, in press, Epub Sep. 14, 2006). Contributing factors to the development of postoperative delirium may include pain severity and central nervous system effects of treatment with opioid analgesics, such as morphine or hydromorphone, commonly used to control pain postoperatively (Leung et al., Anesthesiology A1461, 2005). Inflammation and associated microcirculatory impairment may also contribute to the pathophysiology of postoperative delirium (Hala, supra).

Sepsis-associated delirium and alcohol withdrawal-associated delirium have some clinical and pathophysiologic similarities to postoperative delirium. Systemic inflammation associated with sepsis may lead to brain glial cell activation and accompanying symptoms of delirium (Semmler et al., J. Chem. Neuroanat. 30:144-157, 2005; Mayer, Medicina 58:377-385; Pandharipande et al., Curr. Opin. Crit. Care 11:360-369, 2005). Delirium associated with alcohol withdrawal may also be partly ascribed to brain glial cell activation (Miguel-Hidalgo, Alcohol 41:379, 2006; Valles et al., Brain Pathol. 14: 365, 2004).

Current strategies for management of delirium include treatment with anti-psychotic drugs and sedatives, which have been reported to ameliorate symptoms and improve cognition (Meagher, supra). In recent clinical studies, gabapentin, an anti-epileptic and neuropathic pain drug, which can reduce the need for postoperative opiates, has also been reported to show some efficacy in treatment of delirium (Leung et al., Neurology 67:1251, 2006; Dirks et al., Anesthhesiology 97:560, 2002). However, better methods for treating delirium and improving recovery are clearly needed.

Thus, there remains a need for improved compounds, compositions, and methods of treatment for delirium.

### SUMMARY OF THE INVENTION

In one aspect, the invention provides a method for treating delirium comprising administering to a subject in need thereof a therapeutically effective amount of ibudilast. In one embodiment, the delirium is postoperative delirium. In another embodiment, the delirium is associated withdrawal from a drug. In another embodiment, the delirium is associated withdrawal from alcohol. In another embodiment, the delirium is associated with a metabolic disturbance. In another embodiment, the delirium is associated with sepsis.

In certain embodiments, the subject is human. In certain embodiments, ibudilast is administered systemically, for example, via intravenous, subcutaneous, intraperitoneal, oral, intranasal, sublingual or other systemic routes. In other embodiments, ibudilast is administered centrally, for example, intrathecally. In certain embodiments, multiple therapeutically effective doses of ibudilast are administered to the subject. In certain embodiments, ibudilast is administered according to a daily dosing regimen. In certain embodiments ibudilast is administered once a day or twice a day. In certain embodiments, ibudilast is administered intermittently. In certain embodiments, ibudilast is administered over a timecourse of several days, weeks, or months. In certain embodiments, multiple cycles of treatment are administered to the subject for a time period sufficient to diminish or eliminate symptoms of delirium in the subject.

In certain embodiments, ibudilast is used in combination therapy with one or more other agents for treating delirium. In certain embodiments, one or more agents are selected from the group consisting of analgesics, anti-inflammatory and CNS-partitioning phosphodiesterase inhibitors, antidepressants, neuroleptics, benzodiazepines, and procholinergic agents. Exemplary agents include, but are not limited to, propentofylline, gabapentin, pregabalin, memantine, morphine, hydromorphone, and related opiates, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine. The ibudilast can be administered prior to, concurrent with, or subsequent to one or more other agents for treating delirium. If provided at the same time as another agent, ibudilast can be provided in the same or in a different composition. In one embodiment, ibudilast is administered in combination with gabapentin. In another embodiment, ibudilast is administered in combination with pregabalin. In another embodiment, ibudilast is administered in combination with one or more analgesic opiates. In one embodiment, ibudilast is administered in combination with morphine. In another embodiment, ibudilast is administered in combination with hydromorphone. In yet another embodiment, ibudilast is administered in combination with propentofylline. In certain embodiments, multiple therapeutically effective doses of ibudilast and one or more other agents for treating delirium are administered to the subject. In one embodiment, multiple therapeutically effective doses of ibudilast and gabapentin are administered to the subject. In another embodiment, multiple therapeutically effective doses of ibudilast and pregabalin are administered to the subject. In another embodiment, multiple therapeutically effective doses of ibudilast and an analgesic opiate, such as morphine, hydromorphone, or a related opiate, are administered to the subject. In one embodiment, ibudilast is administered orally and the analgesic opiate is administered intravenously to the subject.

In another aspect, the invention provides a composition comprising ibudilast for treating delirium. In certain embodiments, the composition comprises one or more pharmaceutically acceptable excipients.

In certain embodiments, the composition further comprises one or more other agents for treating delirium. In certain embodiments, one or more agents are selected from the group consisting of analgesics, anti-inflammatory and central nervous system (CNS)-partitioning phosphodiesterase inhibitors, antidepressants, neuroleptics, benzodiazepines, and procholinergic agents. Exemplary agents include, but are not limited to, propentofylline, gabapentin, pregabalin, memantine, morphine, hydromorphone, and related opiates, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine. In one embodiment, the composition comprises a combination of ibudilast and gabapentin. In another embodiment, the composition comprises a combination of ibudilast and pregabalin. In another embodiment, the composition comprises a combination of ibudilast and morphine. In yet another embodiment, the composition comprises a combination of ibudilast and propentofylline.

In yet another aspect, the invention provides a kit comprising ibudilast, and optionally one or more additional agents effective for treating delirium. Ibudilast can be provided in the same or in a different composition as other agents. In certain embodiments, one or more agents are selected from the group consisting of analgesics, anti-inflammatory and central nervous system (CNS)-partitioning phosphodiesterase inhibitors, antidepressants, neuroleptics, benzodiazepines, and procholinergic agents. Exemplary agents include, but are not limited to, propentofylline, gabapentin, pregabalin, memantine, morphine, hydromorphone, and related opiates, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine. In one embodiment, the kit comprises a combination of ibudilast and gabapentin. In another embodiment, the kit comprises a combination of ibudilast and pregabalin. In another embodiment, the kit comprises a combination of ibudilast and morphine. In yet another embodiment, the kit comprises a combination of ibudilast and propentofylline. Instructions (e.g., written, tape, VCR, CD-ROM, DVD, etc.) for administering ibudilast and any other agents usually will be included in the kit.

These and other embodiments of the subject invention will readily occur to those of skill in the art in view of the disclosure herein.

### BRIEF DESCRIPTION OF THE FIGURES

FIG. 1 compares analgesic efficacies of low-dose morphine and ibudilast (AV411) in a rat tail-flick model (see Example 1).

FIG. 2 compares the efficacies of ibudilast and gabapentin, administered either separately or in combination, in attenuating mechanical allodynia in a rat chronic constriction injury (CCI) model of neuropathic pain (see Example 2). Data represent means ±SE. *p<0.05 vs. CCI+Vehicle. *p<0.05 vs. CCI+ibudilast+Gabapentin.

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

### I. DEFINITIONS

In describing and claiming the present invention, the following terminology will be used in accordance with the definitions described below.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

The term “delirium” is defined herein as acute cognitive impairment associated with a medical problem. Delirium may be precipitated by various factors, including but not limited to, surgical trauma, withdrawal from drugs or alcohol, metabolic disturbances, or sepsis. Symptoms of delirium may include hallucinations, delusions, psychosis, disorientation, confusion, anxiety, attention deficit, memory impairment, personality changes, depression, immobility, sleep disturbance, language disturbance, and sensory deficits.

The term “postoperative delirium” is defined herein as acute cognitive impairment following a surgical procedure.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

The term “central nervous system” or “CNS” includes all cells and tissue of the brain and spinal cord of a vertebrate. Thus, the term includes, but is not limited to, neuronal cells, glial cells (astrocytes, microglia, oligodendrocytes), cerebrospinal fluid (CSF), interstitial spaces and the like.

The terms “subject”, “individual” or “patient” are used interchangeably herein and refer to a vertebrate, preferably a mammal. Mammals include, but are not limited to, murines, rodents, simians, humans, farm animals, sport animals and pets.

The term “about”, particularly in reference to a given quantity, is meant to encompass deviations of plus or minus five percent.

The terms “effective amount” or “pharmaceutically effective amount” of a composition or agent, as provided herein, refer to a nontoxic but sufficient amount of the composition to provide the desired response, such as suppression of glial activation, phosphodiesterase activity, or inflammation in the central nervous system of a subject, and optionally, a corresponding therapeutic effect. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation.

By “therapeutically effective dose or amount” of ibudilast, or gabapentin, or other drugs for treating delirium (e.g., glial-attenuators, phosphodiesterase inhibitors, analgesics, antidepressants, neuroleptics, benzodiazepines, and procholinergic agents) is intended an amount that, when ibudilast or gabapentin are administered, either separately or in combination, or when one or more additional drugs are administered as described herein, brings about a positive therapeutic response in treatment of delirium, such as diminishing or eliminating acute or chronic pain or inflammation in a subject and the accompanying symptoms of cognitive impairment.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a novel therapeutic methodology for safely and effectively treating delirium with ibudilast. The methods of the invention attenuate glial cell activation associated with, e.g., surgical trauma, neuropathic pain, alcohol or drug withdrawal, or sepsis, and the accompanying symptoms of delirium.

In order to further an understanding of the invention, a more detailed discussion is provided below regarding methods of treating delirium with ibudilast.

Treatment of Delirium with Ibudilast

In one aspect, the invention provides a method for treating delirium comprising administering to a subject in need thereof a therapeutically effective amount of ibudilast. Ibudilast has been shown in the present application to have modest anti-nociceptive efficacy comparable to low-dose (1.3 mg/kg, s.c.) morphine, as manifested in an acute rat tail-flick model (see Example 1). In addition, ibudilast is not only similarly effective to gabapentin in rat chronic neuropathic pain efficacy, but in combination with gabapentin provides greater pain relief than either agent alone (see Example 2).

Although not wishing to be bound to any particular theory, the following properties of ibudilast may contribute to its efficacy in treating postoperative delirium in humans: 1) ibudilast partitions to the central nervous system, 2) targets some of the underlying pathophysiology associated with delirium (inflammation and reduced blood flow, e.g., cerebral circulation), 3) has both acute and chronic pain efficacy, and 4) reduces the need of postoperative opiates, whose effects on the central nervous system may contribute to postoperative delirium. This constellation of demonstrated properties sets ibudilast apart as a unique potential therapy for post-operative delirium. Moreover, the reported efficacy of gabapentin in treating postoperative delirium, and the evidence presented herein that combinations of ibudilast and gabapentin enhance pain efficacy in a rat pain model compared to treatment with either agent alone (Example 2), indicates that combinations of ibudilast and gabapentin are particularly useful in treating postoperative delirium.

Two other deliriums with some clinical and pathophysiologic similarities to postoperative delirium include sepsis-associated delirium and alcohol withdrawal-associated delirium. Both sepsis-associated delirium and alcohol withdrawal-associated delirium are associated with brain glial cell activation (Miguel-Hidalgo (supra); Valles et al. (supra); Semmler et al. (supra); Mayer (supra)). Ibudilast, which is a glial cell attenuator in vitro and in vivo (see, e.g., A Suzumura et al Brain Res 837:203, 1999; U.S. Patent Application Publication No. 2006/0160843A1, published Jul. 20, 2006; herein incorporated by reference) is therefore expected to be useful in treating sepsis-associated delirium, alcohol withdrawal-associated delirium, or any other type of delirium associated with brain glial cell activation.

In certain embodiments, ibudilast is used in combination therapy with one or more other agents for treating delirium. Such agents include, but are not limited to, analgesics, anti-inflammatory and CNS-partitioning phosphodiesterase inhibitors, antidepressants, neuroleptics, benzodiazepines, and procholinergic agents. Exemplary agents include, but are not limited to, propentofylline, gabapentin, pregabalin, memantine, morphine, hydromorphone, and related opiates, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine. In a preferred embodiment, ibudilast is administered in combination with gabapentin for treating postoperative delirium in a subject. In another preferred embodiment, ibudilast is administered orally and an analgesic opiate is administered intravenously to a subject for treating postoperative delirium.

Pharmaceutical Compositions for Treating Delirium

Ibudilast

Ibudilast is a small molecule drug (molecular weight of 230.3) having the structure shown below.

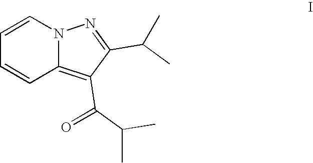

Ibudilast is also found under ChemBank ID 3227, CAS # 50847-11-5, and Beilstein Handbook Reference No. 5-24-03-00396. Its molecular formula corresponds to [C₁₄H₁₈N₂O]. Ibudilast is also known by various chemical names which include 2-methyl-1-(2-(1-methylethyl)pyrazolo(1,5-a)pyridin-3-yl)1-propanone; 3-isobutyryl-2-isopropylpyrazolo(1,5-a)pyridine]; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and the brand name Ketas®. Ibudilast, as referred to herein, is meant to include any and all pharmaceutically acceptable salt forms thereof, prodrug forms (e.g., the corresponding ketal), and the like, as appropriate for use in its intended formulation for administration.

Ibudilast is a non-selective nucleotide phosphodiesterase (PDE) inhibitor (most active against PDE-3, PDE-4, PDE-10, and PDE-11 (Gibson et al. (2006) Eur. J. Pharmacology 538:39-42)), and has also been reported to have LTD4 and PAF antagonistic activities. Its profile appears effectively anti-inflammatory and unique in comparison to other PDE inhibitors and anti-inflammatory agents. PDEs catalyze the hydrolysis of the phosphoester bond on the 3′-carbon to yield the corresponding 5′-nucleotide monophosphate. Thus, they regulate the cellular concentrations of cyclic nucleotides. Since extracellular receptors for many hormones and neurotransmitters utilize cyclic nucleotides as second messengers, the PDEs also regulate cellular responses to these extracellular signals. There are 11 families of PDEs: Ca²⁺/calmodulin-dependent PDEs (PDE1); cGMP-stimulated PDEs (PDE2); cGMP-inhibited PDEs (PDE3); cAMP-specific PDEs (PDE4); cGMP-binding PDEs (PDE5); photoreceptor PDEs (PDE6); high affinity, cAMP-specific PDEs (PDE7); specific PDE (PDE8); high affinity cGMP-specific PDEs (PDE9); and mixed cAMP and cGMP PDEs (PDE10, PDE11).

As stated previously, a reference to any one or more of the herein-described drugs, in particular ibudilast, is meant to encompass, where applicable, any and all enantiomers, mixtures of enantiomers including racemic mixtures, prodrugs, pharmaceutically acceptable salt forms, hydrates (e.g., monohydrates, dihydrates, etc.), different physical forms (e.g., crystalline solids, amorphous solids), metabolites, and the like.

Formulation Components

Excipients/Carriers

Optionally, in addition to ibudilast, the compositions of the invention may further comprise one or more pharmaceutically acceptable excipients or carriers. Exemplary excipients include, without limitation, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose; D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

An ibudilast-containing composition of the invention may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in an ibudilast composition of the invention is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Other Actives

A formulation (or kit) in accordance with the invention may contain, in addition to ibudilast, one or more additional active agents effective in treating delirium. Preferably, the active agent is one that possesses a mechanism of action different from that of ibudilast. Such actives include analgesics, anti-inflammatory and CNS-partitioning phosphodiesterase inhibitors, antidepressants, neuroleptics, benzodiazepines, and procholinergic agents.

Sustained Delivery Formulations

Preferably, the compositions are formulated in order to improve stability and extend the half-life of ibudilast. For example, ibudilast may be delivered in sustained-release formulations. Controlled or sustained-release formulations are prepared by incorporating ibudilast into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, ibudilast can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

Delivery Forms

The ibudilast compositions described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations of the invention are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation of the invention may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

The compositions of the present invention may also be prepared in a form suitable for veterinary applications.

Method of Administration

As set forth above, preferred methods of delivery of ibudilast-based therapeutic formulations for the treatment of delirium include systemic and localized delivery, i.e., directly into the central nervous system. Such routes of administration include but are not limited to, oral, intra-arterial, intrathecal, intramuscular, intraperitoneal, subcutaneous, intravenous, intranasal, and inhalation routes.

More particularly, an ibudilast-containing formulation of the present invention may be administered for therapy by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular neuralgia-associated syndrome being treated, and the specific combination of drugs employed.

One preferred mode of administration for delivery of ibudilast is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J. Virol. 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

A particularly preferred method for targeting spinal cord glia is by intrathecal delivery, rather than into the cord tissue itself.

Another preferred method for administering the ibudilast-based compositions of the invention is by delivery to dorsal root ganglia (DRG) neurons, e.g., by injection into the epidural space with subsequent diffusion to DRG. For example, an ibudilast-based composition can be delivered via intrathecal cannulation under conditions where ibudilast is diffused to DRG. See, e.g., Chiang et al., Acta Anaesthesiol. Sin. (2000) 38:31-36; Jain, K. K., Expert Opin. Investig. Drugs (2000) 9:2403-2410.

Yet another mode of administration to the CNS uses a convection-enhanced delivery (CED) system. In this way, ibudilast can be delivered to many cells over large areas of the CNS. Any convection-enhanced delivery device may be appropriate for delivery of ibudilast. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, an ibudilast-based composition of the invention is delivered via CED devices as follows. A catheter, cannula or other injection device is inserted into CNS tissue in the chosen subject. Stereotactic maps and positioning devices are available, for example from ASI Instruments, Warren, Mich. Positioning may also be conducted by using anatomical maps obtained by CT and/or MRI imaging to help guide the injection device to the chosen target. For a detailed description regarding CED delivery, see U.S. Pat. No. 6,309,634, incorporated herein by reference in its entirety.

An ibudilast composition of the invention, when comprising more than one active agent, may be administered as a single combination composition comprising a combination of ibudilast and at least one additional active agent effective in the treatment of delirium. In terms of patient compliance and ease of administration, such an approach is preferred, since patients are often adverse to taking multiple pills or dosage forms, often multiple times daily, over the duration of treatment. Alternatively, albeit less preferably, the combination of the invention is administered as separate dosage forms. In instances in which the drugs comprising the therapeutic composition of the invention are administered as separate dosage forms and co-administration is required, ibudilast and each of the additional active agents may be administered simultaneously, sequentially in any order, or separately.

Active agents other than ibudilast can be administered by any acceptable route of administration. Agents may be administered using the same or different routes of administration in accordance with any medically acceptable method known in the art. Suitable routes of administration include parenteral administration, such as subcutaneous (sc), intraperitoneal (ip), intramuscular (im), intravenous (iv), or infusion, oral, pulmonary, nasal, topical, transdermal, and suppositories.

Kits

Also provided herein is a kit containing at least one composition of the invention comprising ibudilast and optionally one or more additional agents effective for treating delirium, accompanied by instructions for use. For kits comprising a combination of ibudilast and one or more additional agents effective for treating delirium, ibudilast can be provided in the same or in a different composition as other agents.

For example, in instances in which each of the drugs themselves are administered as individual or separate dosage forms, the kit comprises ibudilast in addition to each of the drugs making up the composition of the invention, along with instructions for use. The drug components may be packaged in any manner suitable for administration, so long as the packaging, when considered along with the instructions for administration, clearly indicates the manner in which each of the drug components is to be administered.

For example, for an illustrative kit comprising ibudilast and gabapentin, the kit may be organized by any appropriate time period, such as by day. As an example, for Day 1, a representative kit may comprise unit dosages of each of ibudilast and gabapentin. If each of the drugs is to be administered twice daily, then the kit may contain, corresponding to Day 1, two rows of unit dosage forms of each of ibudilast and gabapentin, along with instructions for the timing of administration. Alternatively, if one or more of the drugs differs in the timing or quantity of unit dosage form to be administered in comparison to the other drug members of the combination, then such would be reflected in the packaging and instructions. Various embodiments according to the above may be readily envisioned, and would of course depend upon the particular combination of drugs, in addition to ibudilast, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister paks, dessicants, and the like.

Dosages

Therapeutic amounts can be empirically determined and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and particular combination being administered.

Therapeutically effective amounts can be determined by those skilled in the art, and will be adjusted to the requirements of each particular case. Generally, a therapeutically effective amount of ibudilast will range from a total daily dosage of about 0.1 and 200 mg/day, more preferably, in an amount between 0.1 and 100 mg/day, 0.1-60 mg/day, 0.1 and 40 mg/day, or 0.1 and 20 mg/day. Administration can be one to three times daily for a time course of one day to several days, weeks, months, and even years, and may even be for the life of the patient.

Practically speaking, a unit dose of any given composition of the invention or active agent can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Anti-Nociceptive Pain Testing in Rats

Experimental Procedures

Male Sprague-Dawley (CD) rats were acclimated to semi-confined conditions on a heated glass surface (76-78° F.). On the first day, rats were acclimated to a glass surface for 20-30 minutes. Rats were in chambers made of plexiglass and could move freely in either direction. No stimulus was given on day 1. On day 2, rats were acclimated for 5 minutes, and a high intensity beam of light was directed at each rat tail approximately 4 cm from the tip. The time it took for a rat tail to flinch, or move suddenly away from the heat source was recorded as the baseline latent period for tail flick. Three trials were done with 5 minutes between trials for each rat. The light source was moved for the 2nd and 3rd trials one cm to the left and right of the original trial to avoid tissue damage. The procedure performed on day 2 was repeated on day 3 with animals that were first treated with 7.5 mg/kg ibudilast intraperitoneally (i.p.), 1.3 mg/kg morphine subcutaneously (s.c.), or vehicle (1 ml/kg saline, s.c.). The mean latent periods were randomized into 3 groups, equivalent in baseline latency. Animals were dosed and returned to their cages until 5 minutes before each hourly time point, when they were returned to the glass surface and stimulated with the heat source (3 trials/time point). A 35% PEG 400 solution vehicle, used for formulating ibudilast, was also shown not to have analgesic activity and performed like the saline control.

Results

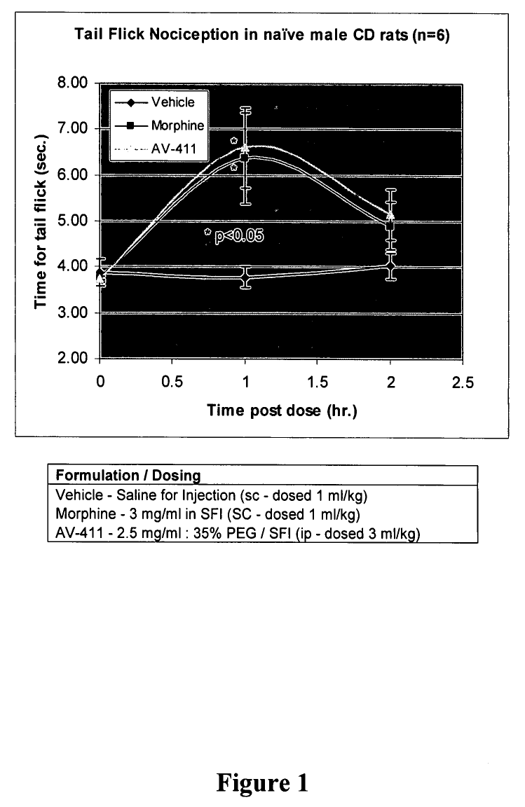

As shown in FIG. 1, administration of ibudilast (7.5 mg/kg, i.p.) provided acute analgesic efficacy (increased the latency time) in the rat tail-flick model with a magnitude and duration similar in nature to that of a relatively low dose of morphine (1.3 mg/kg, s.c.).

### EXAMPLE 2

### Efficacies of Ibudilast and Gabapentin

Experimental Procedures

Pathogen-free adult male Sprague-Dawley rats (250-375 g; Harlan Labs, Madison, Wis., USA) were used in all experiments, except in the spinal nerve ligation study and Irwin test, where male Wistar (Han) rats (160-200 g) were used (Elevage Janvier, Le Genest-Saint-Isle, France). Rats were housed in temperature (23±3° C.) and light-(12 hour: 12 hour light:dark cycle; lights on at 7 AM) controlled rooms with standard rodent chow and water available ad libitum. Behavioral testing and drug administrations were performed during the light cycle.

Ibudilast (Haorui Inc., Chongqing, China; Sigma, St; Louis, Mo., USA) was dissolved in 35% polyethylene glycol (PEG) 400 (Sigma) in sterile physiological saline, except for the tissue distribution study where the vehicle was 15% ethanol in saline. Fresh solutions were made every other day and stored at room temperature (RT). Gabapentin (Neurontin®, Parke-Davis/Pfizer) was formulated in sterile saline at a concentration of 10 mg/ml and stored at RT.

Chronic constriction injury (CCI) was created at mid-thigh level of the left hind leg as previously described (Bennett and Xie, 1988). Four sterile, absorbable chromic gut sutures (cuticular 4-0, 27″; Ethicon, Somerville, N.J., USA) were loosely tied around the isolated sciatic nerve under isoflurane anesthesia (induction 4-5%, maintenance 2-3% in oxygen; Phoenix Pharm., St. Joseph, Mo., USA). The sciatic nerves of sham-operated rats were identically exposed, but not ligated.

The von Frey test (Chaplan et al., 1994) for mechanical allodynia (a type of chronic pain) was performed within the sciatic innervation area of the hind paws as previously described (Milligan et al., 2000). Calibrated Semmes-Weinstein monofilaments (von Frey hairs; Stoelting, Wood Dale, Ill., USA) were applied randomly to left and right hind paws to elicit paw withdrawal responses. The monofilaments used ranged from 3.61 (0.407 g) to 5.18 (15.136 g) bending force. Assessments were made prior to (baseline) and at specific times after surgery or drug injections, as detailed below for each experiment. Behavioral testing was performed blind with respect to drug administration. The behavioral responses were used to calculate the 50% paw withdrawal threshold (absolute threshold), by fitting a Gaussian integral psychometric function using a maximum-likelihood fitting method, allowing parametric statistical analyses (Harvey, 1986; Treutwein and Strasburger, 1999), as described in detail previously (Milligan et al., 2000). In the CCI and SNL experiments, rats were only tested on the ipsilateral hind paw. In the paclitaxel experiments, rats were tested on both hind paws, since systemic paclitaxel treatment results in bilateral allodynia. As thresholds in these rats did not differ between left and right hind paws at any time point in any group, the data are presented as average values from both hind paws.

### RESULTS

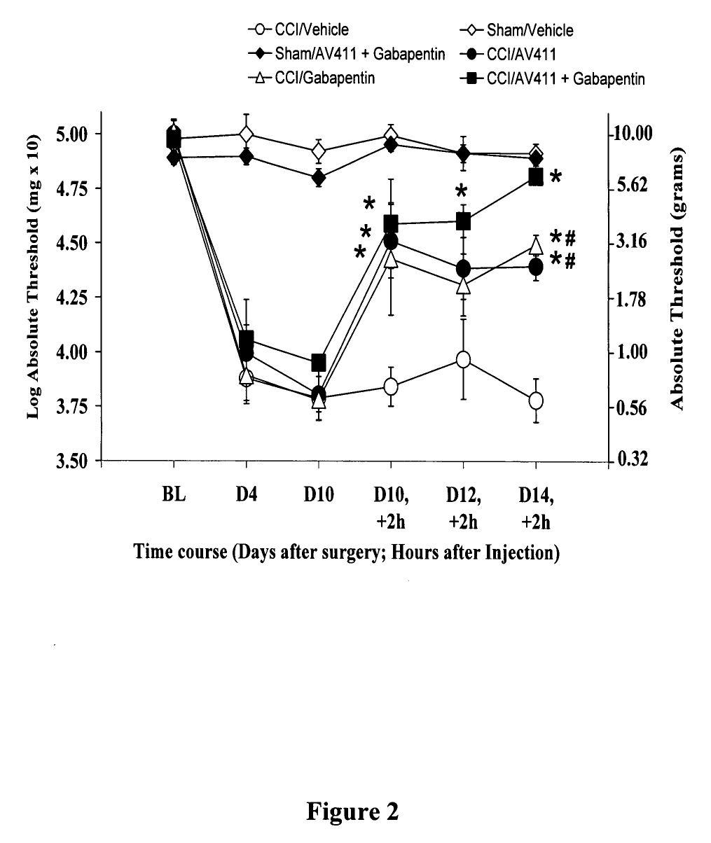

Rats received sham or CCI surgery on Day 0, and were administered vehicle, ibudilast (7.5 mg/kg), gabapentin (50 mg/kg) or both from Days 10-14 post-surgery. Low-threshold mechanical sensitivity was assessed by the von Frey test, before surgery (baseline, BL), and from Day 10-14 before or 2 hours after the morning administration. As shown in FIG. 2, twice daily i.p. ibudilast and i.p. gabapentin had similar efficacy in attenuating CCI-induced mechanical allodynia, and efficacy was enhanced such that pain sensations (mechanical allodynia) were nearly completely resolved when the drugs were combined.

### CONCLUSION

Ibudilast has been shown in the present application to have anti-nociceptive efficacy comparable to low-dose morphine, as manifested in an acute rat tail-flick model. In addition, ibudilast is not only similarly effective to gabapentin in rat chronic neuropathic pain efficacy, but in combination with gabapentin provides greater pain relief than either agent alone.

Although not wishing to be bound to any particular theory, the following properties of ibudilast may contribute to its efficacy in treating postoperative delirium in humans: 1) ibudilast partitions to the central nervous system, 2) targets some of the underlying pathophysiology associated with delirium (inflammation and reduced circulation), 3) has both acute and chronic pain efficacy, and 4) reduces the need of postoperative opiates, whose effects on the central nervous system may contribute to postoperative delirium. Moreover, the evidence presented herein that combinations of ibudilast and gabapentin enhance pain efficacy in a rat pain model compared to treatment with either agent alone (Example 2) indicates that combinations of ibudilast and gabapentin will be particularly useful in treating postoperative delirium.

Although preferred embodiments of the subject invention have been described in some detail, it is understood that obvious variations can be made without departing from the spirit and the scope of the invention as described herein.

All references cited herein, including patents, patent applications and other publications, are hereby incorporated by reference in their entireties.

What is claimed is:

1. A method for treating delirium comprising administering to a subject in need thereof a therapeutically effective amount of ibudilast.

2. The method of claim 1, wherein the delirium is postoperative delirium.

3. The method of claim 1, wherein the delirium is associated withdrawal from a drug.

4. The method of claim 1, wherein the delirium is associated withdrawal from alcohol.

5. The method of claim 1, wherein the delirium is associated with sepsis.

6. The method of claim 1, wherein the subject is human.

7. The method of claim 1, wherein the ibudilast is administered systemically.

8. The method of claim 7, wherein the ibudilast is administered intravenously, subcutaneously, orally, intranasally, or sublingually.

9. The method of claim 1, wherein the ibudilast is administered centrally.

10. The method of claim 9, wherein the ibudilast is administered intrathecally.

11. The method of claim 1, wherein multiple therapeutically effective doses of ibudilast are administered to the subject.

12. The method of claim 11, wherein ibudilast is administered according to a daily dosing regimen.

13. The method of claim 12, wherein ibudilast is administered twice a day.

14. The method of claim 11, wherein ibudilast is administered intermittently.

15. The method of claim 11, wherein multiple cycles of treatment are administered to the subject for a time period sufficient to diminish or eliminate symptoms of delirium in the subject.

16. The method of claim 1, further comprising administering one or more agents other than ibudilast for treating delirium.

17. The method of claim 16, wherein one or more agents are selected from the group consisting of analgesics, anti-inflammatory and central nervous system (CNS)-partitioning phosphodiesterase inhibitors, antidepressants, neuroleptics, benzodiazepines, and procholinergic agents.

18. The method of claim 17, wherein one or more agents are selected from the group consisting of propentofylline, gabapentin, pregabalin, memantine, morphine, hydromorphone, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine.

19. The method of claim 18, wherein a therapeutically effective amount of ibudilast is administered in combination with a therapeutically effective amount of gabapentin to the subject.

20. The method of claim 16, wherein multiple therapeutically effective doses of ibudilast and one or more other agents for treating delirium are administered to the subject.

21. The method of claim 20, wherein multiple therapeutically effective doses of ibudilast and gabapentin are administered to the subject.

22. The method of claim 20, wherein multiple therapeutically effective doses of ibudilast and pregabalin are administered to the subject.

23. The method of claim 20, wherein multiple therapeutically effective doses of ibudilast and hydromorphone are administered to the subject.

24. The method of claim 23, wherein the ibudilast is administered orally and the hydromorphone is administered intravenously.

25. The method of claim 24, wherein the delirium is postoperative delirium.

26. A composition comprising ibudilast and one or more other agents for treating delirium.

27. The composition of claim 26 further comprising a pharmaceutically acceptable excipient.

28. The composition of claim 26, wherein one or more agents are selected from the group consisting of analgesics, anti-inflammatory and central nervous system (CNS)-partitioning phosphodiesterase inhibitors, antidepressants, neuroleptics, benzodiazepines, and procholinergic agents.

29. The composition of claim 28, wherein one or more agents are selected from the group consisting of propentofylline, trazodone, mianserin, chlorpromazine, droperidol, haloperidol, perazin, pimozide, olanzapine, risperidone, lorazepam, diazepam, and physostigmine.

30. The composition of claim 28, wherein the composition comprises a combination of ibudilast and gabapentin.

31. The composition of claim 28, wherein the composition comprises a combination of ibudilast and pregabalin.

32. The composition of claim 29, wherein the composition comprises a combination of ibudilast and propentofylline.

33. A kit comprising ibudilast and instructions for treating delirium.

34. The kit of claim 33, further comprising one or more other agents for treating delirium.

35. The kit of claim 34, wherein one or more agents are selected from the group consisting of analgesics, anti-inflammatory and central nervous system (CNS)-partitioning phosphodiesterase inhibitors, antidepressants, neuroleptics, benzodiazepines, and procholinergic agents.

36. The kit of claim 35, wherein one or more agents are selected from the group consisting of propentofylline, gabapentin, pregabalin, memantine, morphine, hydromorphone, and related opiates, cannabinoids, tramadol, lamotrigine, carbamazepine, duloxetine, milnacipran, tricyclic antidepressants, trazodone, mianserin, chlorpromazine, droperidol, haloperidol, perazin, pimozide, olanzapine, risperidone, lorazepam, diazepam, and physostigmine.

37. The kit of claim 36, wherein the kit comprises a combination of ibudilast and gabapentin.

38. The kit of claim 36, wherein the kit comprises a combination of ibudilast and pregabalin.

39. The kit of claim 36, wherein the kit comprises a combination of ibudilast and propentofylline.

40. The kit of claim 36, wherein the kit comprises a combination of ibudilast and an analgesic opiate.
